(19) United States
(12) Patent Application Publication
(10) Pub. No.: US 2025/0222198 A1
(43) Pub. Date: Jul. 10, 2025

(54) AN ANAL IRRIGATION PROBE

(71) Applicant: Coloplast A/S, Humlebaek (DK)

(72) Inventor: Lars Olav Schertiger, Fredensborg (DK)

(21) Appl. No.: 18/853,455

(22) PCT Filed: Mar. 29, 2023
(86) PCT No.: PCT/DK2023/050071

§ 371 Date: Oct. 2, 2024

(30) Foreign Application Priority Data
Apr. 8, 2022 (DK) PA 2022 70191

(51) Int. Cl. A61M 3/02

(52) U.S. Cl. CPC A61M 3/0283; A61M 3/0295; A61M 2210/1067

(57) ABSTRACT

An anal probe is provided. The anal probe has a handle with a cavity with an absorbing element. The cavity provides a bypass such that air can be vented through the cavity. When liquid reaches the cavity, the absorbing element increases in volume and will close off the bypass.

The invention relates to an anal irrigation probe and an anal irrigation system including an anal irrigation probe.

### BRIEF DESCRIPTION OF THE DRAWING

The accompanying drawings are included to provide a further understanding of embodiments and are incorporated into and a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 illustrates an anal irrigation system as described herein.

FIGS. 2 and 3A, 3B and 3C illustrate cross-sectional views of an example of a probe with a handle as described herein. FIGS. 3A to 3C illustrate close-ups of the handle in three different states from dry to wetted state of an absorbing element as described herein.

FIGS. 4 and 5 illustrate side views of the handle of the FIG. 2 example. In FIG. 4, the handle is seen from the outside with the cavity at the side and in FIG. 5, the handle is seen from the inside.

FIGS. 6 and 7A, 7B illustrate cross sectional views of an example of a probe with a handle as described herein. FIGS. 7A and 7B illustrate close-ups of the handle in dry and wetted state of an absorbing element as described herein.

FIGS. 8 and 9 illustrate cross-sectional views of examples of probes with retention means.

### DETAILED DESCRIPTION

Examples relate to an anal irrigation probe comprising
an insertable portion including a first interior lumen configured for allowing irrigation liquid to flow through the insertable portion
a handle portion including a second interior lumen configured for allowing irrigation liquid to flow through the second interior lumen
   wherein the handle portion includes a cavity communicating with the second interior lumen and the ambience, the cavity holding an absorbing element capable of increasing in volume when transitioning between a dry state and a wetted state, the cavity having a first volume exceeding a second volume of the absorbing element in a dry state, such that air is capable of bypassing the absorbing element when the absorbing element is in a dry state.

Examples relate to an anal irrigation probe configured for auto-priming such that air is vented from the tube through a bypass, the bypass being in the form of a cavity comprising an absorbing element, the cavity being positioned in a handle of the probe, the air being vented from the tube through the bypass prior to liquid reaching the catheter, and wherein the bypass is closed by liquid reaching the cavity causing the absorbing element to increase in volume and thereby close the bypass.

Examples relate to an anal irrigation system comprising a container, tubing, a pump and an anal irrigation probe as described above.

When using an anal probe as described above, the user does not have to think about priming the system prior to use. By priming is meant that the system, in particular the tubing, is emptied from air prior to use, such that the liquid is present at the probe. It may lead to great discomfort for the user if air is pumped into the bowels prior to beginning the irrigation procedure.

In an anal irrigation system as described above, the air is vented out from the probe prior to liquid reaching the probe. An absorbing element as described, will be able to transition between a dry state, in which it has a second volume and a wetted state, in which the volume of the absorbing element is increased to a third volume, which is larger than the second volume. In the dry state of the absorbing element, the air has the possibility of bypassing the absorbing element, because the volume of the cavity (the first volume) is larger than the volume of the absorbing element in dry state, the second volume. This means that when the user has inserted the probe into the rectum and starts to pump, the air from the tubing will be able to leave the probe through the first cavity at the handle and be vented out from the system rather than be transferred into the bowels of the user. The bowels of the user will always have a certain interior pressure exceeding the ambient air pressure and therefore, the air will leave the probe at the position with least pressure, which is through the cavity and to the exterior of the probe (the ambience). When the pump has been activated for a while, the air from the tubing has been vented out and liquid will have reached the handle of the probe. In this state, the absorbing element begins uptake of liquid and quickly increases in size until it blocks the bypass possibility through the cavity. The absorbing element may increase in volume until it fills the cavity. In this state, the wetted state of the absorbing element, the bypass is blocked and liquid from the tubing will pass through the second interior lumen of the handle into the first interior lumen of the insertable portion and from there exit the probe at a proximal portion of the probe.

In the following, whenever referring to a proximal end of end element described herein, the referral is to the end closest to the user of the system. Whenever referring to the distal end of an element, the referral is to the end opposite of that. This means that the insertable portion and in particular the exit opening of the probe is in a proximal portion of the probe. The handle of the probe is in a distal portion of the probe. The interior lumens of the handle and insertable portion of the probe extends in an axial direction. The direction transverse to the axial direction may be referred to as the transverse direction.

An irrigation system typically comprises a reservoir or container for irrigation liquid, an anal probe and tubing connecting those two. The system will also include a pump for pumping the irrigation liquid into the intestines. The pump may be a manual pump or an electric pump. The pump may be configured for pumping air into the container to pressurise it, and thus allow liquid for being displaced from the container. The pump may be a liquid pump configured to directly displace the liquid from the container.

The anal probe comprises an insertable portion, which is configured for being inserted into the rectum or stoma of a user, and a handle portion, which is configured for being held by the user during insertion and which is not configured for being inserted into the rectum. The two portions may be separated by a transversely extending flange.

In a proximal portion of the insertable portion, the probe is provided with at least one exit opening for letting irrigation liquid leave the probe and enter into the bowels of a user during use. The at least one exit opening may be an open end of the insertable portion. Alternatively, the proximal end of the insertable portion may be closed and the exit opening may be in the form of at least one, typically two transversely extending eyelet(s).

The handle portion may be configured for being connected to the tubing and may comprise a connector for connecting to a tube-connector. The handle portion may also have the tubing directly welded to it.

The absorbing element may be made from a superabsorber such as is well-known in the art of wound dressings. These superabsorber materials are typically in the form of either particles, and is thus known as SAP, or in the form of fibres, known as SAF.

The absorbing element may be made from a hydrocolloid material, such as an alginate, CMC or polysaccharide.

The absorbing element may be made of foam material, for example a PU-foam.

The absorbing element may be in a dry state, in which the absorbing element has a second volume. The absorbing element may be in a wetted state, in which the absorbing element has a third volume, which is larger than the second volume. By wetted state is meant that the absorbing element has absorbed liquid. The cavity has a first volume, which in examples is 20% larger than the second volume of the absorbing element in a dry state. The third volume of the absorbing element is in examples as large as the first volume of the cavity—and maybe even slightly larger, such that the absorbing element extends slightly into any inlets to the cavity or causes an inner mesh-material to deform slightly inwards. It is contemplated that the absorbing element will be able to close the bypass, if the absorbing element fills approximately 95% of the first volume.

In other words, in a dry state, the first interior lumen and the second interior lumen forms a flow path communicating with a reservoir and wherein the cavity provides a bypass to the flow path. In a wetted state, this bypass is closed.

The anal irrigation probe may be configured for auto-priming. In the context of this disclosure, auto-priming refers to automatic priming, meaning that the user does not have to think about priming the probe for use, but rather that the probe comprises features allowing for or configured for auto-priming.

In examples, the cavity is positioned at an exterior wall of the handle.

In examples, the cavity is positioned juxtaposed to the second interior lumen. This means that the handle is provided with an extra inlet channel leading to the cavity holding the absorbing element.

In examples, the cavity communicates with the ambience through one or more outlets in an exterior wall of the handle.

In examples the cavity communicates with the ambience through a porous foil material.

When the probe is primed, air will enter into the cavity and has to be provided with an exit from the cavity to the ambience. This may be done by a variety of means; a couple of examples are mentioned above. It is an advantage, if the absorbing element is somewhat protected from being torn during handling of the probe; therefore, outlets in an exterior wall of the handle is an advantage. However, a porous foil will in most situations provide enough of a barrier to protect the absorbing element.

In examples, the handle has a partition wall dividing the second interior lumen from the cavity. This partition wall faces the interior lumen of the handle (the second interior lumen) and is in examples provided with a number of inlets to the cavity. This means that the partition wall may form part of the handle and may be moulded together with the handle. The inlets may be provided in the moulding process.

In examples the partition wall comprises or is in the form of a mesh-material, which may be inserted into the handle either during or after manufacturing of the handle.

The partition wall may be perforated as described, either by providing inlets or by providing a mesh-material, and these perforations provide the possibility of flow of air from the interior lumen of the handle (the second interior lumen) through the perforations to the cavity and from there to the ambience.

In examples, the partition wall is not perforated. Instead, the handle comprises a distal inlet to the cavity in addition to the distal inlet to the second interior lumen.

The absorbing element is capable of absorbing liquid fast, such as within approximately 1-2 seconds. During this absorbing process, the absorbing element will increase in volume and is capable of increasing its volume at least twice and maybe even three or four times. In examples, the absorbing element comprises superabsorber. Superabsorber is well-known in the art of wound dressing and diapers and has a capability of increasing its volume many times during absorption. As examples, the superabsorber may comprise polymers of acrylic acid or carboxymethyl cellulose. In examples the absorbing element may be a foam element and in examples the foam element may comprise a suberabsorber.

In examples, the absorbing element comprises a layer of foil on an exterior facing surface. This will prevent the absorbing element from accidentally absorbing any liquid from the exterior through the exit from the handle to the exterior, which, as described above, may be in the form of outlets or a porous foil.

In an example, the anal irrigation probe is provided with retention means. In an example, the retention means is in the form of an inflatable balloon. In an example, the retention means is in the form of a foam element. The foam element on the catheter may be as described in the international application no. PCT/DK2021/050343. When a retention means in the form of a foam element is used, it may be an advantage that the user does not have to prime the probe prior to insertion. This is because, the foam element may change its characteristics upon contact with liquid—and this transformation is in an optimal situation only taking place after the probe has been positioned in the rectum of a user. A probe as referred to in the international application mentioned above, is in some examples wrapped in a liquid-dissolvable film. In particular in this case, it is an advantage, if the priming is only done when the probe is inserted into the rectum.

### DETAILED DESCRIPTION OF THE DRAWING

Initially, it shall be noted that the figures are schematic illustrations intended only to address the principles and functions of the anal probe described herein and are not to be considered limiting to the scope of the attached claims. Furthermore, the figures and particularly the individually illustrated elements are not necessarily to scale, neither individually nor in relation to each other.

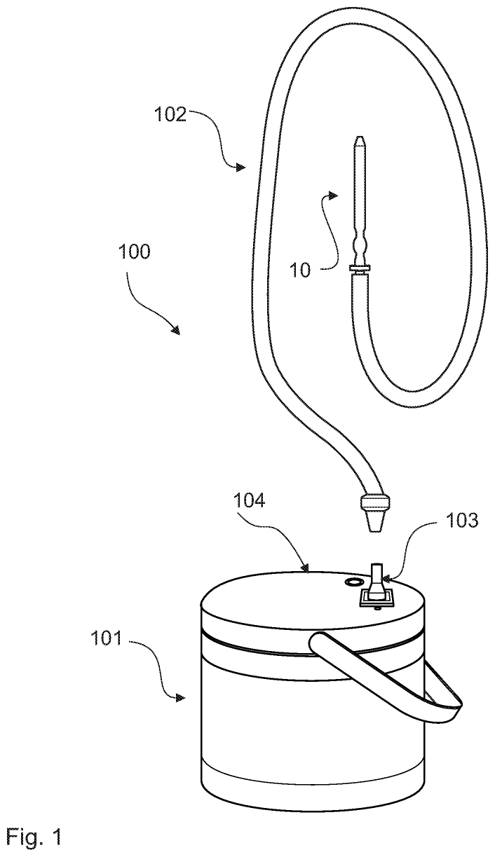

FIG. 1 illustrates an anal irrigation system 100. The system comprises a container for irrigation liquid 101, a tube 102 connected to an anal probe 10. A connector 103 connects the container 101 to the tube 102. In the example of the system illustrated in FIG. 1, the connector 103 is positioned in the lid 104 of the container. The container 101 may have other features, such as a handle, a scale, and so forth. The description here is mostly related to the anal probe 10, which will be described in more detail below.

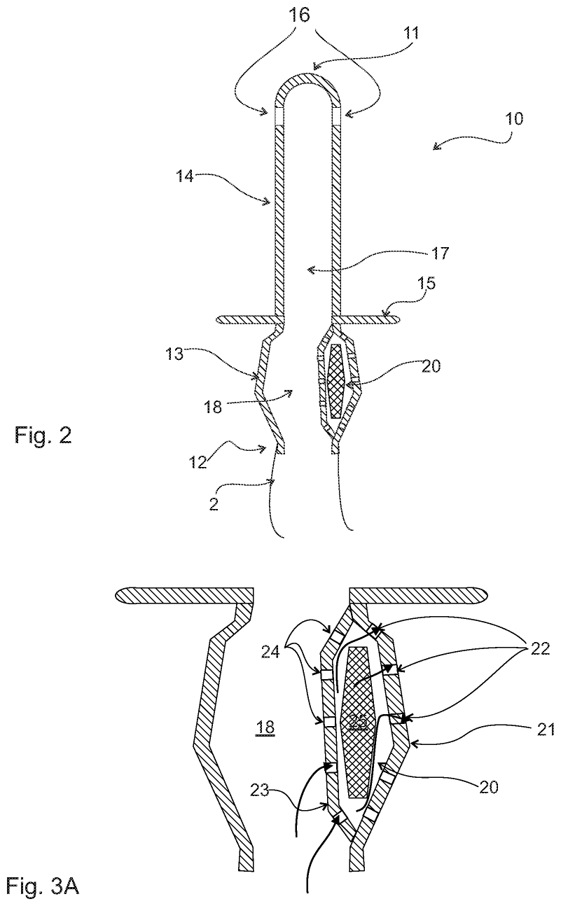

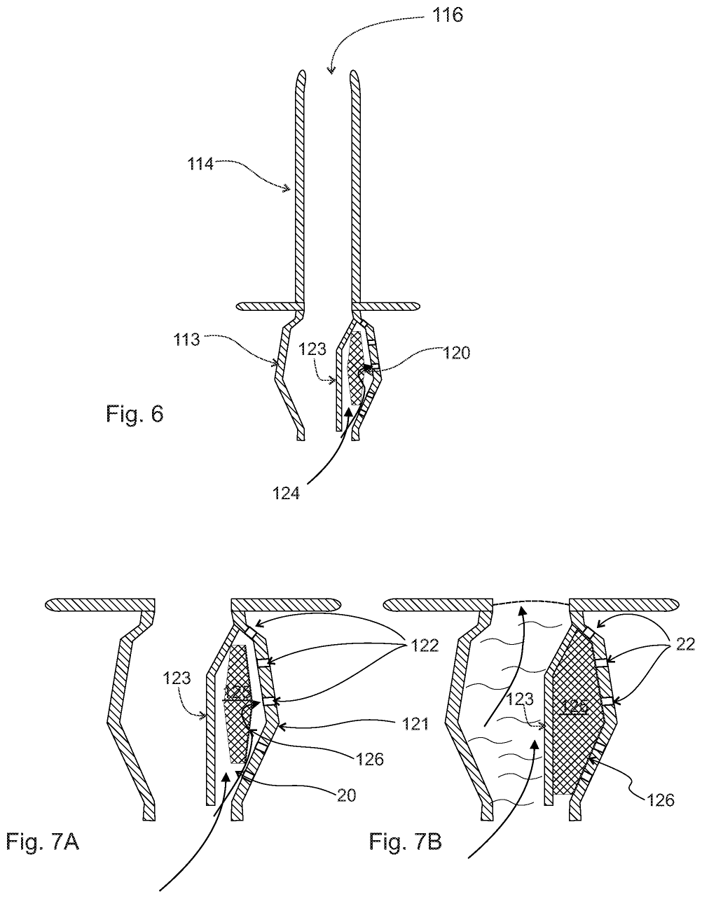

FIG. 2 illustrates an anal probe 10 as described herein. The probe 10 has a proximal insertion end 11 and a distal end 12. The distal end of the probe is provided with a handle 13, which in the illustrated example is integral with the insertable portion 14 but separated therefrom by a flange 15. The flange 15 may be used to function as a hold for the user, when the probe is to be inserted. The probe is in the illustrated example provided with two eyelets 16 in a proximal portion of the probe. These two eyelets 16 function as exit-openings to provide flow-communication between a first interior lumen 17 in the insertable portion 14 and the exterior of the probe, so as to allow irrigation liquid to exit the probe through the eyelets. Other examples (see FIG. 6) relate to the probe having an open proximal end, such that irrigation liquid can exit through the end. However, a closed proximal end and transversely extending eyelets may provide for a better distribution of irrigation liquid in the bowels and may be perceived as being less traumatic for the intestinal wall.

The handle 13 is provided with a second interior lumen 18, communicating with a lumen of the tube (see FIG. 1) and the first interior lumen 17, so as to provide a flow-path for the irrigation liquid from the reservoir, through the tube and through the second interior lumen of the handle and the first interior lumen of the insertable portion of the probe and finally exit the probe through the eyelets. The handle 13 has a cavity 20 communicating inwardly with the second interior lumen and outwardly with the ambience.

FIGS. 3A to 3C illustrate close-ups of the handle in cross-sectional view. At the position of the cavity 20, the handle exterior wall 21 is provided with a number of outlets 22. A partition wall 23 of the handle, may be provided with a number of inlets 24 to the cavity. Alternatively, the partition wall 23 may be made of mesh material, such as non-woven or the like. For the example illustrated in FIGS. 3A to 3C, where there is no distal inlet into the cavity, it is a requirement that the partition wall 23 has to be permeable for liquid and air. The cavity 20 has an absorbing element 25 inside it. The absorbing element is capable of absorbing liquid and will increase in volume during absorption—see FIG. 3B, the arrows indicate an increase in volume. The function of the cavity 20 with the absorbing element 25 and the air-venting valve function will be explained with particular reference to FIGS. 3A to 3C.

In FIG. 3A, the absorbing element 25 is illustrated in its dry state. In this state, the volume the volume V1 of the cavity (the first volume) is larger than the volume V2 of the absorbing element in a dry state, the second volume. This is shown because there is a free volume surrounding the absorbing element 25 in the cavity. When the probe is to be prepared for use, the user will insert the probe with the handle attached into the rectum. The flange 15 functions as a hold and a stop, ensuring that the user does not accidentally insert the handle 13 into the rectum. Prior to use, the system, for example the tubing, might include entrapped air. When the user initiates the irrigation procedure, the entrapped air will flow through the system towards the probe “in front of” the irrigation liquid. The cavity 20 with the absorbing element will function as an air-valve allowing this entrapped air to leave the probe prior to entering into the insertable portion of the probe and through the eyelets into the rectum of the user. This is due the volume V2 of the absorbing element 25 being smaller than the volume V1 of the cavity 20, when the absorbing element 25 is in its dry state. This is illustrated in FIG. 3A, where the arrows indicate how air passes through the permeable partition wall 23 into the cavity 20 and exits the handle through the outlets 22 in the handle exterior wall 21. In FIG. 3B, it is illustrated how the liquid front L has reached the cavity 20 and how liquid begins flowing through the partition wall 23 into the cavity 20 and the absorbing element 25 increase in volume, as indicated by the small arrows. During this uptake or absorption of liquid, the absorbing element 25 will increase in volume until it completely fills the cavity 20, as indicated in FIG. 3C. The absorbing element will in this position have reached its third volume V3, which is the volume in a wetted state of the absorbing element. In this position, the cavity is closed and liquid passing through the second interior lumen 18 of the handle is unable to leave the handle through the cavity. Thus, the liquid can only pass into the insertable portion 14, through the first interior lumen 17 and exit the probe through the eyelets 16.

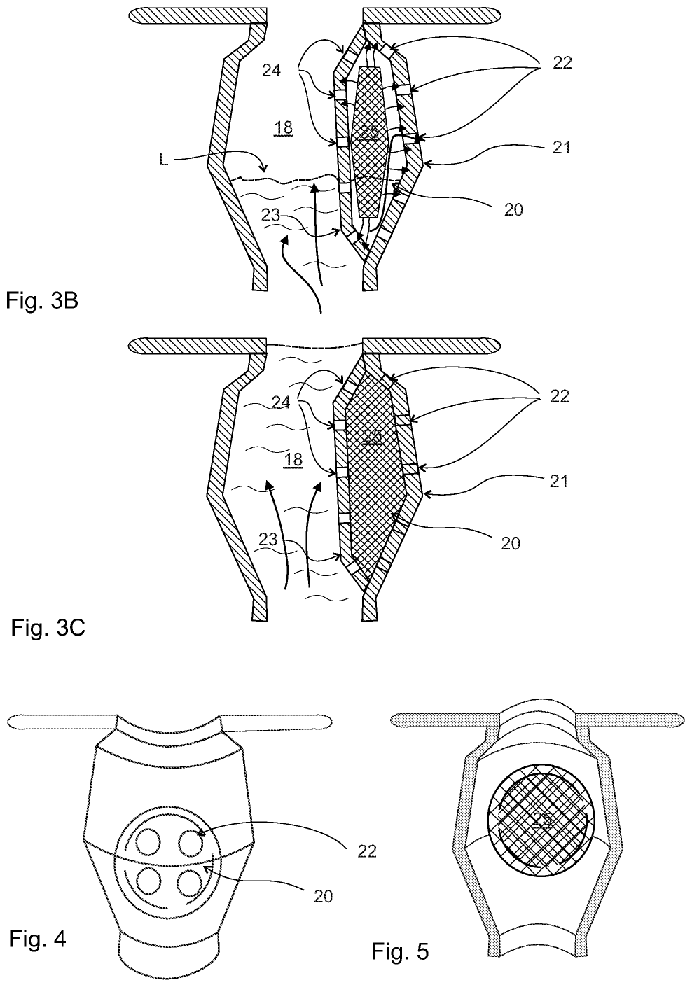

FIG. 4 illustrates the handle seen from the exterior, where the outlets 22 can be seen in the exterior wall 21 of the handle. The cavity 20 and the absorbing element 25 inside it are indicated by dotted lines. FIG. 5 illustrates the handle seen from the interior, where the permeable partition wall 23 is indicated as a mesh. The cavity 20 and the absorbing element 25 are also illustrated.

FIGS. 6 and 7A, 7B illustrate another example of a probe 110 with a handle 113. The insertable portion 114 differs from the example illustrated in FIG. 2 in that in this example, the insertable portion 114 has an open proximal end providing the exit opening 116. Furthermore, the cavity 120 differs. In the example of FIGS. 6 and 7A, 7B, the cavity 120 is formed as an interior closed channel of the handle with an inlet 124 at a distal end of the handle. Thus, as opposed to the permeable partition wall of the handle shown in the example of FIGS. 2 to 5, this example of FIGS. 6 and 7 includes a non-permeable partition wall 123 in the handle. The partition wall of the handle is in this example not permeable to air/liquid, as there is an inlet 124 in the distal end ensuring the passage of air/liquid to the cavity 120. The handle is also in this example provided with outlets 122 in an exterior wall 121 of the handle. An absorbing element 125 is provided in the cavity and functions as described earlier and illustrated in FIG. 7B, where the absorbing element 125 completely closes of the outlets 122 from the cavity. In the example illustrated in FIGS. 6, 7A and 7B, the absorbing element 125 is provided with an exterior facing foil element 126. This will protect the absorbing element 125 form accidentally absorbing any liquid from the exterior through the outlets 122 prior to use of the probe.

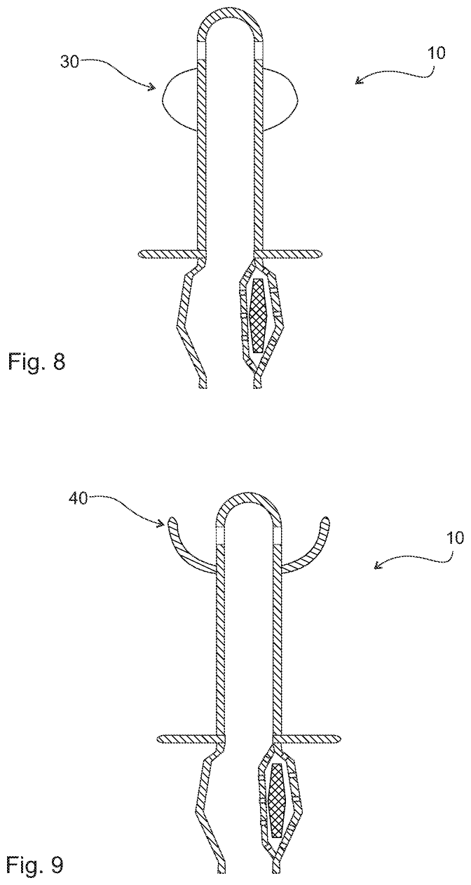

FIGS. 8 and 9 illustrate examples of a probe 10 similar to the one in FIG. 2. The only difference is that in the examples of FIGS. 8 and 9, the probe is provided with a retention element. In FIG. 8, the retention element is in the form of an inflatable balloon 30, which is illustrated in the inflated state. In FIG. 9, the retention element is in the form of a foam element 40, which is illustrated in the expanded state.

Embodiments, and features of the various exemplary embodiments described in this application, may be combined with each other (“mixed and matched”), unless specifically noted otherwise.

1. An anal irrigation probe comprising:
a proximal insertable portion including a first interior lumen configured for allowing irrigation liquid to flow through the insertable portion and out of an eyelet formed in a proximal end of the proximal insertable portion,
a distal handle portion including a second interior lumen configured for allowing irrigation liquid to flow through the second interior lumen and into the first interior lumen;
wherein the distal handle portion includes an exterior wall and a partition wall disposed in the second interior lumen and attached to an interior surface of the exterior wall of the distal handle portion to form a cavity,
an absorbing material disposed in the cavity, with the absorbing material having a first volume in a dry state and a second volume in a wetted state, with the second volume larger than the first volume and the cavity having a cavity volume exceeding the first volume of the absorbing material in the dry state,
an inlet formed in the partition wall and communicating with the absorbing material disposed in the cavity; and
an outlet formed in the exterior wall of the distal handle portion to expose the absorbing material to ambient atmosphere.

2. The anal irrigation probe of claim 1, wherein the anal irrigation probe is configured for auto-priming where air entering the second interior lumen of the distal handle portion is vented through the inlet formed in the partition wall and out of the outlet formed in the exterior wall of the distal handle portion to provide an air bypass; and
wherein the air bypass is closed to air passage by a portion of the irrigation liquid entering the cavity and causing the absorbing even material to transition to the wetted state and increase from the first volume to the second volume and thereby close the bypass.

3. (canceled)

4. (canceled)

5. (canceled)

6. The anal irrigation probe of any claim 1, wherein the outlet formed in the exterior wall of the distal handle portion is one of a plurality of outlets formed in the exterior wall of the distal handle portion, and the plurality of outlets is provided by a porous foil.

7. (canceled)

8. (canceled)

9. The anal irrigation probe of claim 1, wherein the inlet formed in the partition wall comprises a mesh-material porous to air and liquid.

10. The anal irrigation probe of claim 1, wherein the inlet formed in the partition wall comprises a distal inlet formed in the partition wall and communicating with the cavity.

11. The anal irrigation probe of claim 1, wherein the absorbing material comprises superabsorber comprising one of a polymer of acrylic acid and a carboxymethylcellulose.

12. The anal irrigation probe of claim 1, wherein the absorbing material comprises a layer of foil on an exterior facing surface of the absorbing material.

13. (canceled)

14. The anal irrigation probe of claim 1, wherein the anal irrigation probe further comprises an inflatable balloon that is inflatable to retain the anal irrigation probe in a rectum of a user.

15. The anal irrigation probe of claim 1, wherein the anal irrigation probe further comprises a foam element that is inflatable to retain the anal irrigation probe in a rectum of a user.

16. The anal irrigation probe of any of the preceding claims, wherein the cavity volume is 20% larger than the first volume of the absorbing material in the dry state.

17. (canceled)

18. (canceled)
